(19) United States
(12) Patent Application Publication
(10) Pub. No.: US 2007/0106127 A1
(43) Pub. Date: May 10, 2007

(54) Automated patient monitoring and counseling system

(76) Inventor: Brian M. Alman, Leucadia, CA (US)

Correspondence Address: CHARMASSON, BUCHACA & LEACH, LLP, 1545 HOTEL CIRCLE SOUTH, SUITE 150, SAN DIEGO, CA, 92108-3426, US

(21) Appl. No.: 11/248,949

(22) Filed: Oct. 11, 2005

(51) Int. Cl. A61B 5/00; A61B 5/02

(52) U.S. Cl. 600/300; 128/905; 128/920; 600/485; 600/500

(57) ABSTRACT

An automated, computerized system for vicariously and remotely monitoring the conditions of a patient, providing professional counseling, and coaching the patient into sound behavior and health-enhancing activities. A computerized central installation receives, over a public communication network, patient's vital sign measurements such as blood pressure, glucose level and pulse rate. The measurements are gathered at the patient's residence by a battery of instruments, polled by the central installation at scheduled intervals, automatically checked against preset limits and analyzed to generate diagnostics and assessments of patient's condition. The diagnostics and assessments are used to selectively retrieve from a data bank of prerecorded messages, feedback advice, past performance charts and motivating audiovisual-programs. If warranted, health professionals are alerted so that they can intervene and contact the patient directly. The communications between the central installation and the patient are scripted by a computer program routine to mimic a natural telephone conversation.

### FIELD OF THE INVENTION

This invention relates to information dispensing systems accessible via public communication networks, and to remote monitoring, gathering and analysis of critical measurements. More specifically, the invention relates to remote monitoring of a patient's conditions and interactive communication of health-enhancing advice over a communication network system.

### BACKGROUND

In the field of health care delivery, one of the most expensive components is the physician intervention and time which sometimes necessitates regrettable but practically necessary curtailment of certain types of followup care such as the monitoring of a patient's medication intake, and mental attitude. Indeed, few Health Management Organizations (HMO's)will authorize consultation with a physician or even with auxiliary medical professionals to establish and maintain life style modification such as changes of diet, exercise patterns, and stress reduction. Yet, these types of followup treatments have been shown beneficial to victims of disease such as , stroke, diabetes, asthma, chronic pain, depression, addiction and some forms of cancer.

It has been demonstrated that a patient suffering from one of the above listed conditions and a variety of other ailments, have responded very well to been coached by a psychologist into life style modifications, including changes of diet, and exercise patterns. Persons recovering from surgical procedures such as heart bypass surgery, for example, are required to make life style changes in order to improve their chance of long term survival.

The prior art offers various types of automated therapeutic behavior modification programs vicariously provided by a computerized installation via telephone or other communication networks to a plurality of persons, as exemplified by the disclosures in U.S. Pat. No. 6,849,045 Iliff, U.S. Pat. No. 6,607,784 Suzuki et al, U.S. Pat. No. 6,039,688 Douglas et al., and U.S. Pat. No. 5,596,994 Bro. In most of these systems, prerecorded therapeutic and motivating messages from health care professionals are stored and classified in a library, then communicated to the patient in response to some detected or perceived condition.

Each one of the prior art systems specializes in dealing with a particular disease or condition, and does not offer a comprehensive approach to automatic condition monitoring and therapeutic counseling that is flexible enough to accommodate a wide range of conditions and situations, and use scientifically proven condition analyzing methods and therapeutic intervening responses.

The present invention is an attempt to take advantage of technological breakthroughs in the field of computer sciences, remote monitoring devices, and communication systems to provide lifelike consultations with virtual health care professionals economically and at all hours of the day. A system according to the invention can be used by a patient to make routine followup calls in order to inquire about medication dosage and side effects, report worrisome developments in their conditions, and request positive motivating feedback to their concerns without direct involvement by a live professional.

### SUMMARY

The invention uses a computerized system to analyze diagnostic parameters received from a remote site over a public communication network and to feed back prerecorded therapeutic messages to patients suffering from a wide range of physiological and psychological conditions. The prerecorded messages from a trained counselor are enhanced by multimedia productions and graphical information. Patients' stations are called on a regular basis to gather healthcare information and assist them in complying with the healthcare regimen prescribed by their therapists. The health care information is parsed to identify physiological parameters such as blood pressure and blood sugar levels generated by monitoring devices, and psychological parameters such as depression and anxiety levels elicited through scripted colloquies between the patient and a central automatic data processing installation computer. Wireless technology is used to collect the physiological data from patients in their own homes, and to transmit that information to a central installation. The collected information is processed in order to constantly update therapeutic interventions, based on patients' needs.

Computer program routines automatically analyze physiological and psychological data from patients and compose feedback messages to (1) monitor the physiological and psychological health of the patient, (2) conduct therapeutic interventions over the patient's cell phone, (3) evaluate the impact of those interventions, and (4) alert healthcare professionals when a patient's condition assessment indicates the need for human intervention.

At the core of the invention are computer programs for collecting personal information assessing its significance, and scripting the system response. A data assessment formula combines physiological and psychological state determinants, and pain level in order to generate program branching commands or instructions to retrieve appropriate feedback messages or a new cycle of inquiries. The physiological determinants are obtained by weighting ranges of measured vital signs parameters with importance and severity coefficients. The psychological determinants are derived from answers elicited from the patient by the program. The answers are obtained and evaluated using Minnesota Multi-phasic Personality Inventory (MMPI) procedures, then weighting the scored parameters by importance and severity coefficients. The pain level is derived from the patient answers to pointed questions formulated by a conversation-scripting program.

The assessment codes and determinants are stored chronologically for each patient so that patient progress or regress can be determined over a period of treatment. Depending on the range of values of diagnostic information analyzed, script generator programs initiate a series of interactive communications, therapeutic interventions, and outcome assessments, utilizing hundreds of prerecorded digital messages stored digitally in a random-access recorded message library. Extreme diagnostics, alert a healthcare professional that personal intervention is required.

Conversations are conducted either orally and preferably by means of the patient's cell phone, or electronically by way of text typed on a personal computer keyboard or numerical pad, or by way of clicking manipulations on a manual entry device such as a mouse.

The complete intercourse between the installation and the patient are scripted to resemble a casual colloquy by a computer program that manipulates and string together a selection of conversational exchange protocols according to multiple choice answers offered to the patient and to program branching instructions triggered by findings from the personal data analysis operation. Therapeutic treatments are based on existing and emergent concepts and principles of positive psychology. Although these concepts and principles are available in the public domain, their application to an automated real-time general behavior monitoring and management system constitute one of the principal advantages of the invention.

Although the following preferred embodiment of the invention is described in connection with the treatment of patient affected with a number of specific physiological and psychological ailments, it should be understood that the invention may be adapted to other applications such as the vicarious coaching of athletes, the corrections of substance abuse, and many other activities that require close monitoring and instant feedback of advice.

### BRIEF DESCRIPTION OF THE DRAWING

FIG. 1 is an block diagram of an automated patient-monitoring and counseling system according to the invention;

FIG. 2 is a organizational diagram of an health care institution including said system;

FIG. 3 is a block diagram of the systems automatic data processing installation;

FIG. 4 is a flow diagram of the conversation scripting process;

FIG. 5 is a block diagram of the prerequisite diagnostic protocol;

FIG. 6 is a flow diagram of the initial consultation process;

FIG. 7 is a diagram of the diagnostic parameter rating;

FIG. 8 is a flow diagram of a pain management consultation process;

FIG. 9 is a flow diagram of a therapeutic intervention process;

FIG. 10 is a flow diagram of a relaxation coaching process;

FIGS. 11 and 12 is a flow diagram of a medication assessment process;

FIGS. 13 and 14 is a flow diagram of interacting diagnostic processes;

FIG. 15 is a block diagram of the computer program structure;

FIG. 16 is a set of physiological and physiological parameters coefficient tables; and

FIG. 17 is a compilation of a particular case evaluation data.

### DESCRIPTION OF THE EXEMPLARY EMBODIMENT(S)

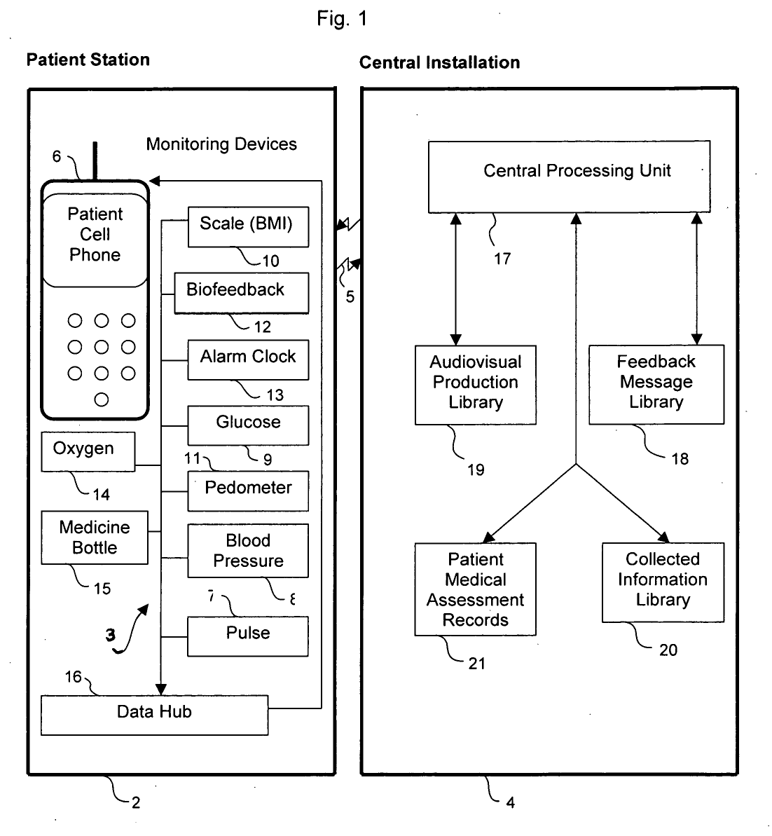

Referring now to the drawing, there is shown in FIG. 1 the basic components of an automated patient monitoring and counseling system 1 according to the invention.

The system comprises a number of remote stations 2 in patients' homes, each station grouping personal vital signs and activity monitoring devices 3, and a central computerized automatic data processing installation 4 that includes a number of hardware equipment and software programs. The stations and the installations are linked over a communication network 5. The communication network may consist of a public system such as the Internet or a private network, and includes a multimedia interface device 6 in each station preferably a cell phone or a personal computer or other similar communication appliance.

The vital sign monitoring devices preferably include a pulse rate monitor 7, a blood pressure monitor 8, a blood glucose level monitor 9, and a personal scale 10. Not shown in the drawing are other useful types of vital sign monitoring devices such as thermometers, blood oxygen monitors, electrocardiograms and other instruments that have been or may be developed in the future. The patient activity monitoring device may comprise a pedometer 11, a stress biofeedback sensor 12, an alarm clock transducer 13 that reports the patient's waking up time, and oxygen supply sensor 14 that reports on the patient's use of an oxygen bottle, and a medicine bottle transducer 15 that tracks the number or amount of pills or other medication taken by the patient.

The monitoring devices usually are of the type that generates digitally coded output signals collected by a home Data Hub 16. Analog signals generated by a monitoring devices may be encoded into digital signals by the Data Hub. The signals are uploaded to the central installation 4 when the station is polled on schedule by the central installation as will be explained below. For example, the blood pressure and pulse monitors may be a person-worn, wireless device that communicates with a data-gathering hub such as the one commercially available under the brand name ADVANCE BPM from Triage Wireless. This is an ambulatory monitor that measures blood pressure, pulse oxymetry, and heart rate continuously, and wirelessly sends information to a web-based system. Other commercially available monitoring devices include A&DE ENGINEERING UA-767 PBT blood pressure monitor, AVANT 4000 Digital Pulse Oxymetry System, NIKE MP3 pedometer, LIFESOURCE UC-321 body weight scale, AUTOGENIC AT53 dual channel EMG biofeedback device, and CYNGUS noninvasive blood glucose meter.

Preferably, the patient's cell phone 6 with capability of sending and receiving full-motion audio/video production, serves as the most visible link between the patient and his virtual counselor at the central installation 4. The principal components of the central installation comprise an automatic data processing unit 17 supported by a prerecorded Feedback Message library 18, and Audiovisual Production library 19, a Collected Information library 20, and a Medical Assessment Record file 21.

The prerecorded Feedback Message library 18 holds hundreds of prerecorded messages from the patient's counselor that have been numerically coded and stored for real time random access playback as needed. The prerecorded messages allow the system to communicate with the patients using natural language voices of real individuals. The ability to simulate a casual colloquy or conversation between the patient and his virtual counselor is a key factor in the therapeutic efficiency of the system. The Audiovisual Production library 19 holds a repertory of segments from movies and television shows that can be selected according to the wishes or taste of the patients and playback as a form of soothing entertainment in situations of stress and anxiety.

The Collected Information library 20 holds the archival records of patients' personal data which includes all the diagnostic parameters and voice-elicited information.

The patient Medical Assessment Record files 21 keeps a chronological account of the patient's overall physical and mental assessment codes which can be scanned to detect progressive or regressive changes and trends during specific periods of a therapeutic treatment program.

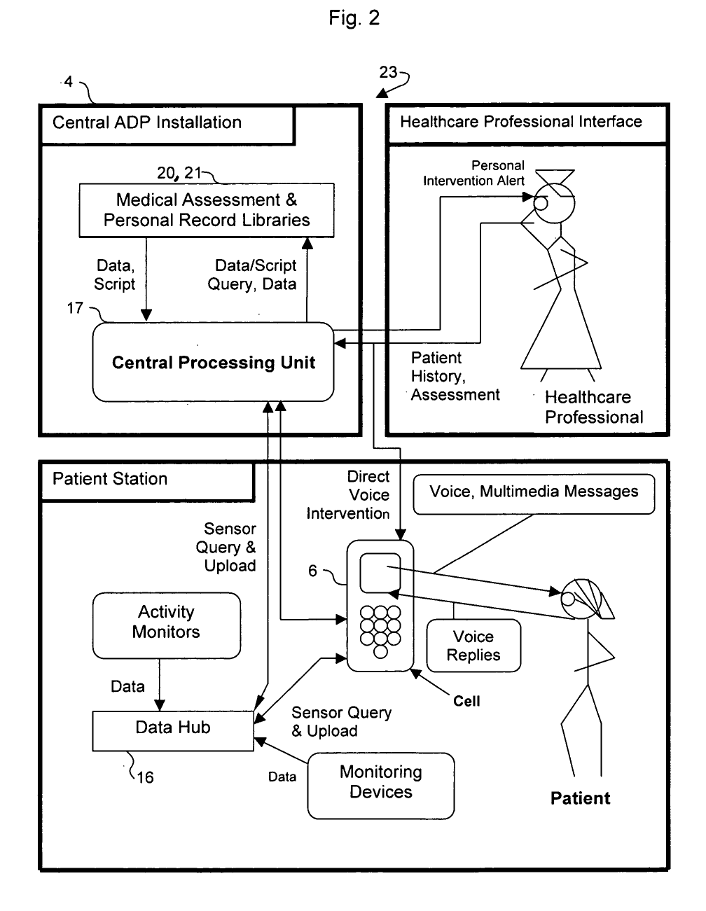

As shown in FIG. 2, the system 1 is part of a comprehensive physical and mental health care providing organization 23. The central automatic data processing installation 4 is backed by health care professionals from one or more medical institutions 24 who can intervene when the system has identified a critical or otherwise alarming patient's condition. Although the patient's station 2 is illustrated with a cell phone 6 for multimedia intercourse between patient and central installation, it should be understood that this multimedia interface could be implemented with a personal computer or any other multimedia communication equipment.

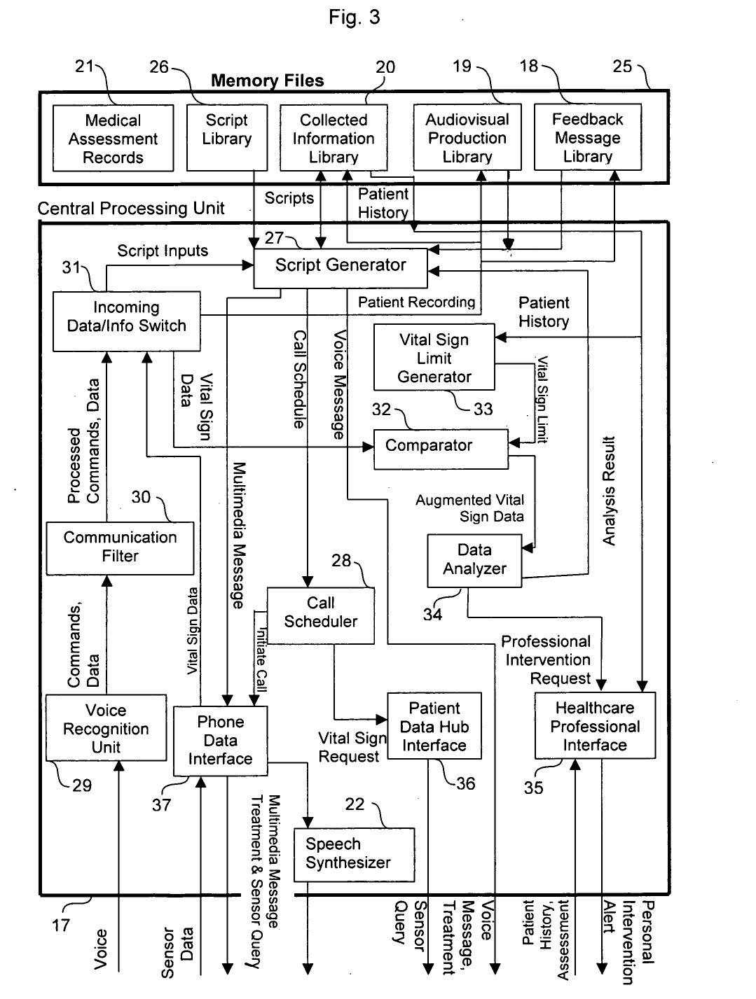

FIG. 3 provides a general block diagram of the central installation structure and functions. In addition to the database and libraries already described, the memory files 25 includes a Script library 26 containing various templates that specify the major sequences of the telephone counseling session with a patient as will be further described below.

The Central Processing Unit (CPU) 17 is shown containing several programs that implemented functions which will be briefly described at this point.

The Script Generator 27 draws upon the memory files 25 to orchestrate the activities that make up the data-gathering and counseling sessions. The Script Generator checks the Collected Information library 20 for scheduled counseling session times. At the appointed date and time, the Script Generator prompts the Call Scheduler 28 to send a diagnostic parameter request to the patient station Data Hub 16. The status and activity data is downloaded from the Data Hub for analyzing and storing into the CPU. While the data is being analyzed or later on at a scheduled time, the Call Scheduler 28 initiates a phone call to the patient.

The patient's verbal replies to queries formulated by the Script Generator 22 are processed by the Voice Recognition unit 29. A Communication Filter 30 separates pure data that can be quantified and digitally coded from statements that are not quantifiable and flags them appropriately. All the data and information are sent to the Incoming Data/Info Switch 31.

The switch directs the received data and information to the appropriate memory files. For instance, the diagnostic measurement data will be sent for storage to the Collected Information library 20 while a audio/visual clip recorded by the patient of his or her own person's demeanor will be stored in the Audiovisual Production library for future playback to the patient. The switch also sends digitally coded vital sign diagnostic measurements to the Comparator 32 to be checked against preset critical limits established by the Limit Generator 33. The limit generator takes into account the patient's history extracted from the Collected Information library 20 to establish alarm limits. The results of the comparison are sent to the data analyzer 34 along with other information such as pedometer readings and noncritical answers.

If a critical condition is detected, professional intervention is requested through the Healthcare Professional Interface unit 35.

Program branching instructions are fed to the Script Generator 21 from the incoming Dat/Info Switch 31 and by the data analyzer 34 depending upon aspects of the conversation or data analyzing results.

The call scheduler 28 communicates with the patient Data Hub 16 through a patient Data Hub Interface 36, and with the patient's cell phone through the phone data interface unit 37.

When the Script Generator 27 communicates with the patient, it makes use of two basic forms of messages. The first are voice messages that are retrieved from the Feedback Message library 18, these messages may be queries or therapeutic interventions. The second form of message is a multimedia message retrieved from the Audiovisual Production library 19. Multimedia messages may include graphical information, sound bites and audiovisual bites. The Script Generator orchestrates the use of these messages to simulate a casual conversation with a patient, using concept and principles of positive physiology. The Script library 26 provides conversation templates of various lengths adapted to various types of interventions.

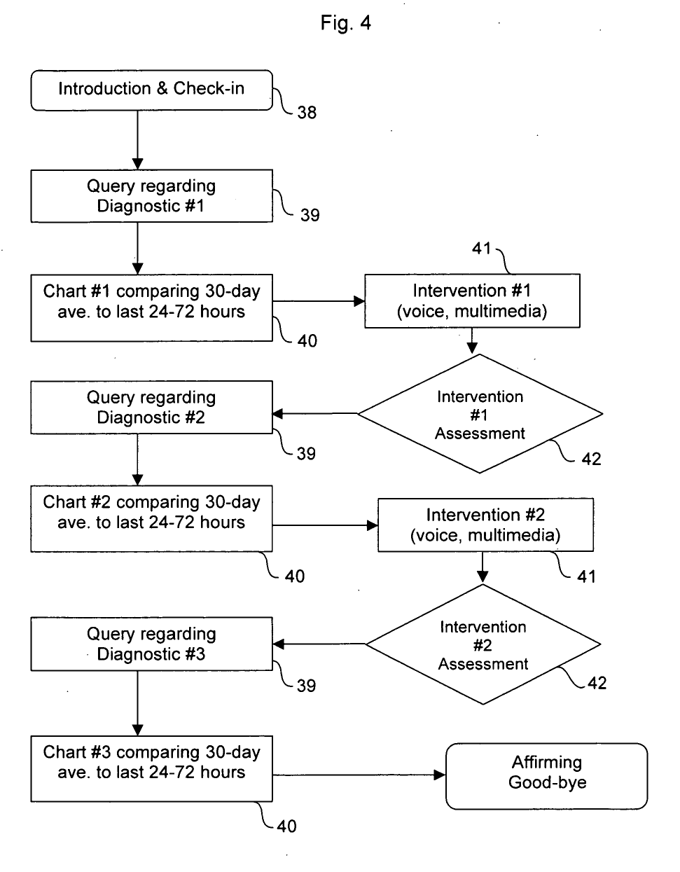

A Speech Synthesizer 22 may be used to translate some of the numerical information extracted from one of the memory files 25 into audible numbers during a phone conversation. FIG. 4 displays a generic scripting routine that controls the sequencing of interactions in the telephone conversation with the patient. This routine is one of many that draws upon the Script library database 26.

The typical conversation begins with an prerecorded introduction 38 from the patient's counselor that asks if the previously scheduled time for the consultation is still convenient to the patient. If not, the consultation is rescheduled. The generic script routine consists of three queries or “polls” of the patient, seeking verbal diagnostics for data that cannot be collected via the monitoring instruments 3 (e.g., psychological diagnostics). The power of the system counseling consultations comes from its ability to organize and present to the patient diagnostic and therapeutic information in an entertaining fashion. Thus, most queries 39 from the installation to the patient are followed by a display of diagnostic information in the form of charts 40 related to the query. For example, queries from the virtual counselor (prerecorded) are followed by graphical displays and verbal explanations of changes in such patient activities and conditions as walking, blood pressure, and pulse, as well as self-reports of pain.

In addition to the charts that provide feedback on changes in various diagnostics, a typical consultation includes two or more therapeutic interventions 41, carefully crafted to help train and motivate patients to comply with their doctors' regimens, in order to achieve their healthcare goals. For each intervention, the system seeks feedback from the patient, assessing 42 whether the intervention was helpful or not.

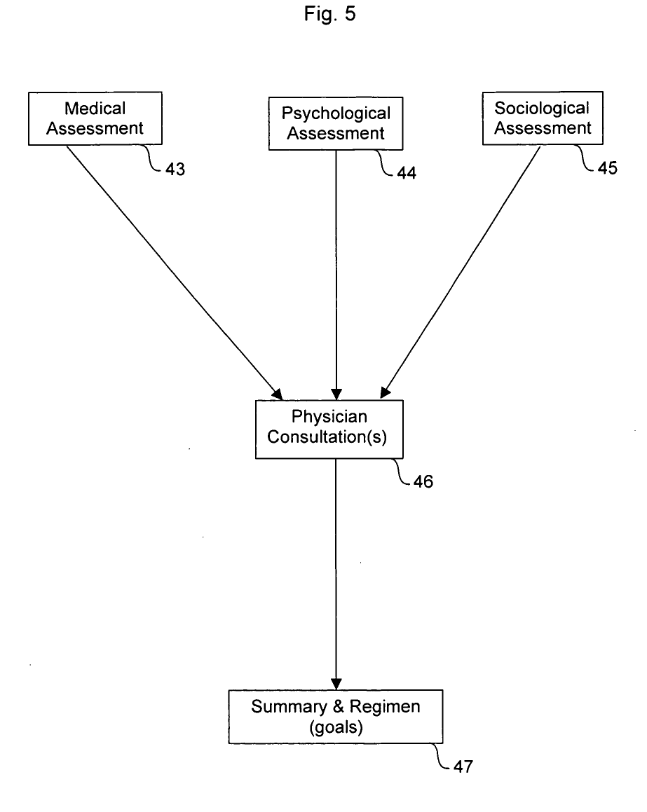

A patient's involvement with the system services does not begin with a telephone call nor does the system operate in isolation from the patient's other healthcare providers. The system works in conjunction with the patient's primary care physician to provide ongoing follow-up for patients with chronic medical conditions that require psycho-social as well as biomedical support on a continuing basis. A prerequisite diagnostic protocol is used to assess the goals of the System program for each patient enrolled as shown on FIG. 5.

A full medical assessment 43 of the patient's health is a prerequisite to an efficient use of the system. The medical assessment should not be limited to the customary examinations of a routine medical check up, but should be also include flexibility and strength assessment, lifestyle assessment, and a nutritional consultation.

In addition to the medical assessment, the Minnesota Multi-phasic Personality Inventory (MMPI) is used to provide a psychological profile 44 of the patient. The system continually assesses anxiety, depression, health concerns, anger, and self-esteem.

A sociological assessment 45 may also be performed using the Adverse Childhood Experience (ACE) diagnostic in order to assess family related issues that may contribute to the patient's chronic medical conditions. Health risk behavior and disease in adulthood are related to the breadth of exposure during childhood to emotional, physical, or sexual abuse, as well as household dysfunction during childhood. These adverse experiences during childhood are linked to multiple risk factors for several of the leading causes of death in adults.

The primary care physicians should hold one or more consultations 46 with the patient to assess the nature of the chronic medical conditions and prescribes a regimen to address those conditions. The regimen may include, but is not limited to, medications, physical therapy, exercise, diet, and complete treatment plans. The regimen seeks to achieve specific goals for the patient, such as pain management.

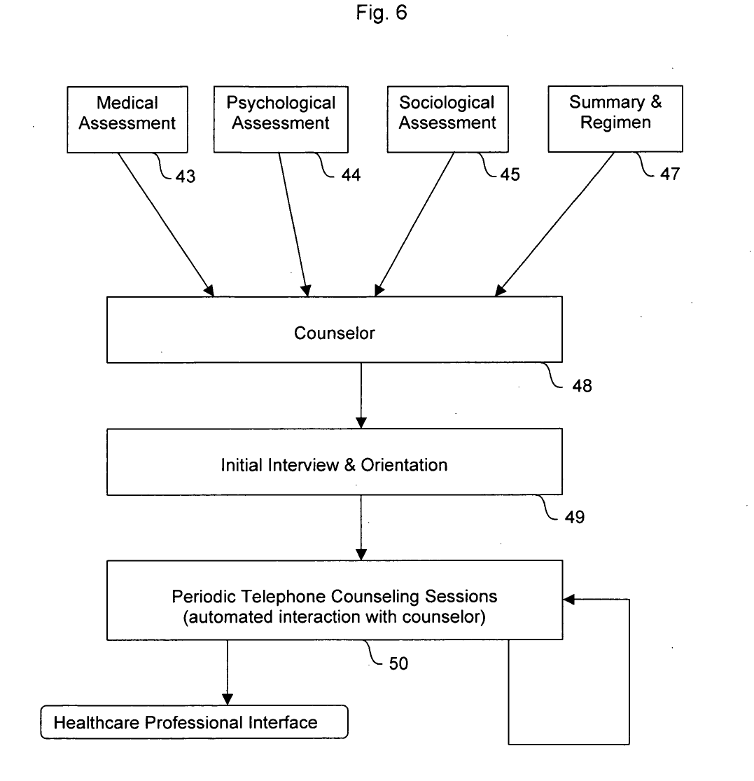

As illustrated in FIG. 6, all of the medical, psychological, and sociological assessment data is transferred from the physicians to a designated professional counselor associated with the organization 23 operating the system. In addition, the physicians also provide the system with the regimen and goals 47 for the patient. The counselor reviews 48 this information and, with the assistance of the technical staff, archives the information in the system computer.

The counselor then speaks with the patient directly by phone. The initial consultation 49 consists of an interview, as well as an orientation session about the services provided by the system. The patient will continue to interact with the counselor indirectly through the prerecorded messages that system will transmit periodically or on demand 50 to the patient on an ongoing basis.

As noted above, system backs up its routine automated patient care with human 51 intervention on an as-needed basis. When healthcare conditions for the patient create emergency or urgent need for direct intervention, the system contacts the appropriate healthcare professional.

The basic logic of the invention is that each patient has a set of physiological and psychological characteristics that are in a constant state of flux. Sometimes changes in physiological conditions (e.g., reduced blood pressure) and psychological conditions (e.g., increased optimism about managing pain) indicate a change for the better. The system uses a series of psychological interventions to promote desired changes in physical and psychological states. These interventions are woven into the fabric of the colloquy between the patient and prerecorded, interactive messages of the patient's counselor.

To monitor a wide range of disparate diagnostics, the system parses the patient's personal data into physiological parameters (php) and psychological parameters (psp), then converts all diagnostic parameters into a common, universal severity coefficient (Sc) in ascending order of severity in a total range from 1.0 (optimal) to 5.0 (dangerously critical), with many gradations therebetween. Basically, the total span of each parameter is divided in a number of subsequent ranges, and each range is assigned a coefficient.

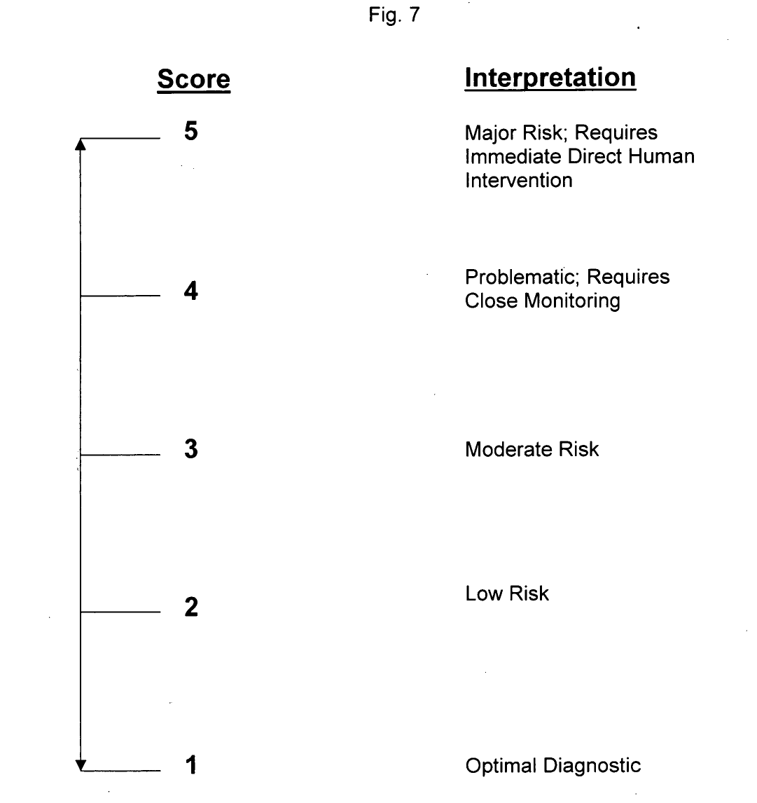

As indicated in FIG. 7, an assignment of a blood pressure severity coefficient at or near five (5) characterizes a systolic reading of 220 and a diastolic reading of 120, or extremely low readings of 90 and 50 respectively. Either case presents a major risk requiring immediate or nearly immediate intervention by a healthcare professional. Generally, these characteristics are physiological characteristics, such as extremely high blood pressure. However, some psychological diagnostics (e.g., suicidal ideation) also can register scores of five. Most psychological indicators, however, do not have associated scores of four (4) or five (5).

At the other end of the standardized severity coefficients are scores at or near one (1). These are optimal diagnostic readings, which apply equally to physiological and psychological diagnostics.

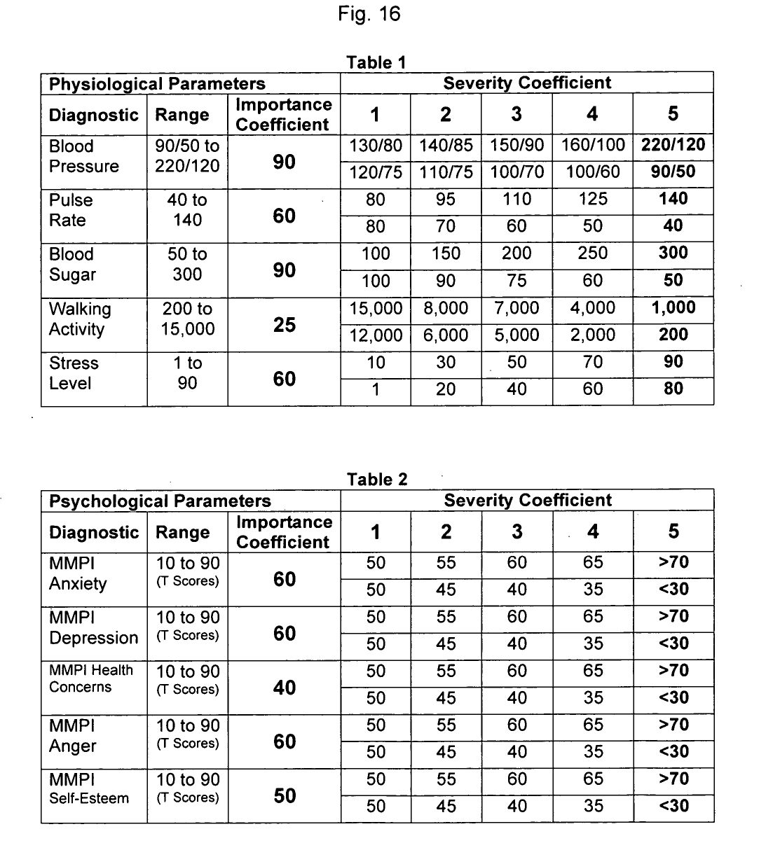

The tables of FIG. 16 display several of the most common diagnostic parameters, the range of values that those parameters assume, and their conversion into the standardized coefficients. Not all parameters have the same weight when analyzing the health of a patient or in contributing to the well-being of the patient. Thus, for every diagnostic, an importance coefficient (Ic) is also indicated in the tables.

When the system computer analyzes the various psychological interventions to implement-and in what sequence-the importance coefficient indicates which characteristics of the patient are most important. The importance coefficient is a number that ranges from 1 to 100, with higher scores indicating greater weight or importance of that diagnostic. Thus, the System seeks to deal with the most critical conditions first by analyzing (1) the importance of the condition (importance coefficient) and (2) the severity of the condition (severity coefficient).

The weights and severity coefficients in FIG. 16 are used by the Data Analyzer 34 to compute either the physical state determinant (PS) or the mental state determinant (PM) for each diagnostic according to the following formulae:

$$PS = {\sum }_{php 1}^{phpx} Ic \times Sc , and PM = {\sum }_{psp 1}^{pspx} Ic \times Sc$$

where: A=Assessment Code

PS=Physical State

PM=Mental State

PL=Pain Level

php=physical parameter

psp=psychological parameter

Ic=Importance Coefficient

Of particular importance to the target patent population served by system is the degree of pain that the patient is experiencing. Pain Level (PL) is assessed in real time by the system, using the following scale:

Pain Level Assessment Scale

| 100 | 200 | 300 | 400 | 500 |
|---|---|---|---|---|
| None | Mild | Medium | Strong | Unbearable |

This information is obtained from the patient through a series of queries over the patient's cell phone. Verbal responses are converted into numeric codes.

By itself, a physical state determinant(PS), a mental state determinant (MS)or pain level (PL) provides only a partial diagnosis of the patient's overall condition. For a more comprehensive assessment of the patient, both the physical state determinants and psychological state determinants are summed to arrive at a assessment core (A) according to the following formula:
A=PS+PM+PL
For example, a hypothetical patient, Kay is a 68-year-old woman who had suffered significant adverse childhood experiences and a failed marriage. She currently suffers from chronic depression, obesity, diabetes mellitus, and coronary artery disease.

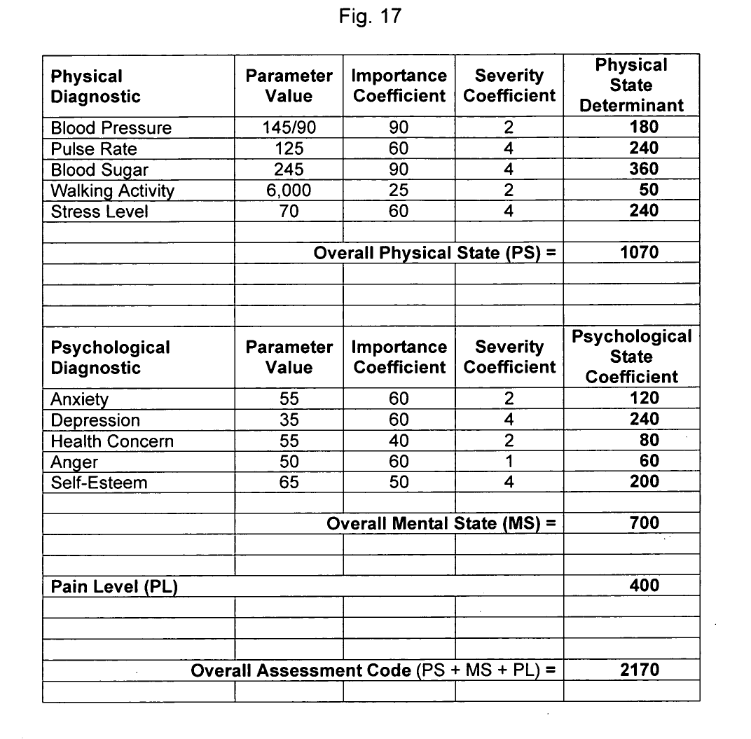

Table 3 of FIG. 17 displays hypothetical values for key physical low risk and psychological parameters, as captured by system through automatic polling from and real-time queries during the phone consultation. Although blood pressure is slightly elevated and walking activity is less than ideal, both parameters are in the range (see FIG. 7). However, pulse rate, blood sugar, and stress levels have severity coefficients in a problematic range that requires close monitoring. Because blood sugar is a critical physiological diagnostic (importance coefficient=90), this diagnostic generates the most elevated (problematic) Physical State Determinant score.

When summed, Kay's Overall Physical State Assessment score is 1070.

Regarding Kay's psychological or mental state, Table 3 indicates that she has low risk severity coefficients for anger and health concerns and an optimal score on the anger diagnostic.

However, Kay is feeling depressed, with a severity coefficient that indicates that her depression requires close monitoring. Also her self-esteem severity coefficient requires close monitoring.

Kay's Overall Mental State is 700.

To assess depression, the system utilizes an abbreviated version of the Beck Depression Inventory, a 21-item measure of depression widely used in clinical practice.

The program elicits the required information from the patient by submitting very focused inquiries, and suggesting a limited number of responses.

Each of the 21 items in the Beck Depression Inventory is framed as follows for the item measuring sadness:

Score Which best describes you right now?

0 I do not feel sad.

1 I feel sad.

2 I am sad all the time and I can't snap out of it.

3 I am so sad or unhappy that I can't stand it.

To assess depression, system displays the above multiple choice menu on the screen of the patient's cell phone, asking to patient to press the number that best describes her sadness.

Once the response is captured by the Phone Data Interface 37, the next item from the Beck Depression Inventory is displayed on the cell phone screen:

Score Which best describes you right now?

0 I get as much satisfaction out of things as I used to.

1 I don't enjoy things the way I used to.

2 I don't get any real satisfaction out of anything anymore.

3 I am dissatisfied or bored with everything.

In this manner, system uses real-time responses from Kay to assess current depression.

In a similar manner, the system program assesses anxiety, health concerns, anger, and self-esteem. Although these psychological diagnostics are widely used with paper-and-pencil tests, the system has adapted them for use with its automated technology.

In addition to the physical and psychological diagnostics, Kay indicates that she is experiencing a great deal of pain (pain level=400).

By summing Kay's Overall Physical State score, her Overall Mental State score, and her Pain Level score, her Overall Assessment Code is calculated as 2170. The computations are made by the Data Analyzer 34. As shown in FIG. 3, the personal information consisting of monitoring device data and responses to voice queries is routed to the Data Analyzer through the Phone Data Interface 37, the Incoming Dat/Info Switch 31, and the Comparator 32. Once the Data Analyzer has made the calculations, the Overall Assessment Code is routed as a program branching instruction to the Script Generator 21. The Script Generator then initiates a set of therapeutic interventions, drawing on the Script library 26 for a protocol responsive to this Overall Assessment Score.

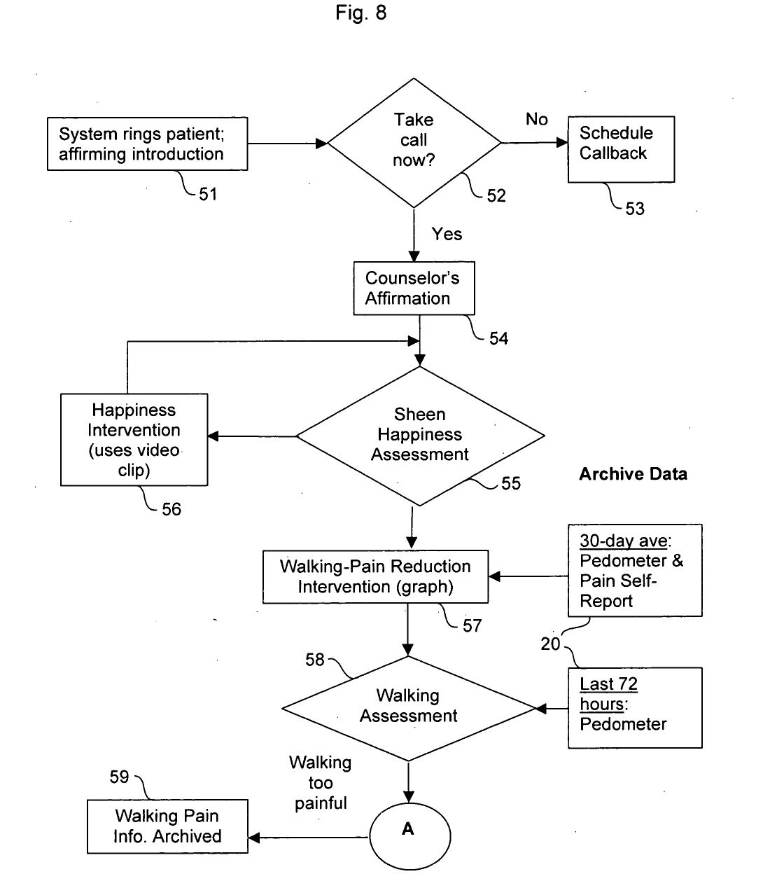

To understand how the system works in greater detail, the overall script of a typical telephone consultation with patient Kay is diagramed in FIGS. 8-10. This is a macro-level analysis, meaning that these diagrams specify the sequencing of many sets of diagnostics and interventions with a patient during a single telephone consultation. In a subsequent section, a detailed analysis is provided of a single diagnostic/intervention sequence.

As shown in FIG. 8, the system computer dials 51 the phone number of the patient and ascertains, through the Voice Recognition unit 29, whether the patient would like to take 52 the consultation call now. If not, the telephone consultation is rescheduled 53. The first action taken according to the program is an initial affirmation 54 by the counselor (“This is Dr. A. with a very caring call to help you in all the ways your doctor described . . . ”). This type of intervention uses specific verbal, visual, and audiovisual messages transmitted to the patient in order to motivate her into following the regimen prescribed by her physician, and to improve her psychological well-being. Positive psychology, as this field of research and practice has been called, has flourished during the last five years. As a result of these efforts, newly created classifications of character strengths and virtues have been added to the Diagnostic and Statistical Manual (DSM) of Mental Disorders by the American Psychiatric Association. Interventions from positive psychology can supplement traditional biomedical interventions to relieve suffering.

The second action dictated by the process in this particular consultation is the Sheen Happiness Assessment 55, an interactive probe using the counselor's prerecorded voice to elicit a self-assessment by the patient (“How is your overall health today, Kay?”). The program analyzes the response (“Okay”), recognizes that oral answer and generate processible data in the form of a numeric score. Alternatively, the system may ask the patient to rate her overall health on a one to five scale. This score can be compared to prior scores generated in previous telephone consultations. Based on the patient's response, the program initiates a personalized happiness intervention 56, utilizing a comedic film clip of an actress that the patient likes. The video clip retrieved from the Audiovisual Production Library 19, and displayed on the screen of the cell phone, lasts about 30 seconds.

After the video clip, the system dictates another assessment of the patient's happiness, using a different prerecorded probe. If the patient indicates an interest, another comedic video clip of the same actress is displayed on the patient's cell phone.

Next, the system displays on the patient's cell phone a graph 57, retrieved from the Collected Information Library 20, indicating the patient's self-report of pain and her degree of walking from a week ago. The pedometer 11 has monitored the patient's walking, 24 hours a day, seven days a week. This information is collected in digital form and uploaded to the patient's station Data Hub 16 in her home. When queried, the pedometer data are then uploaded to the installation 4, where they are archived in the Collected Information Library 20. This archived data is used to initiate an intervention, because recent data, e.g., during the last 72 hours, indicates a reduction in the patient's walking. The goal of the intervention is to show the patient that her pain level subsides with exercise. After displaying the graph, the system probes 58 why the patient has decreased her walking. She reports that walking is “too painful.” This information is archived 59 for future use.

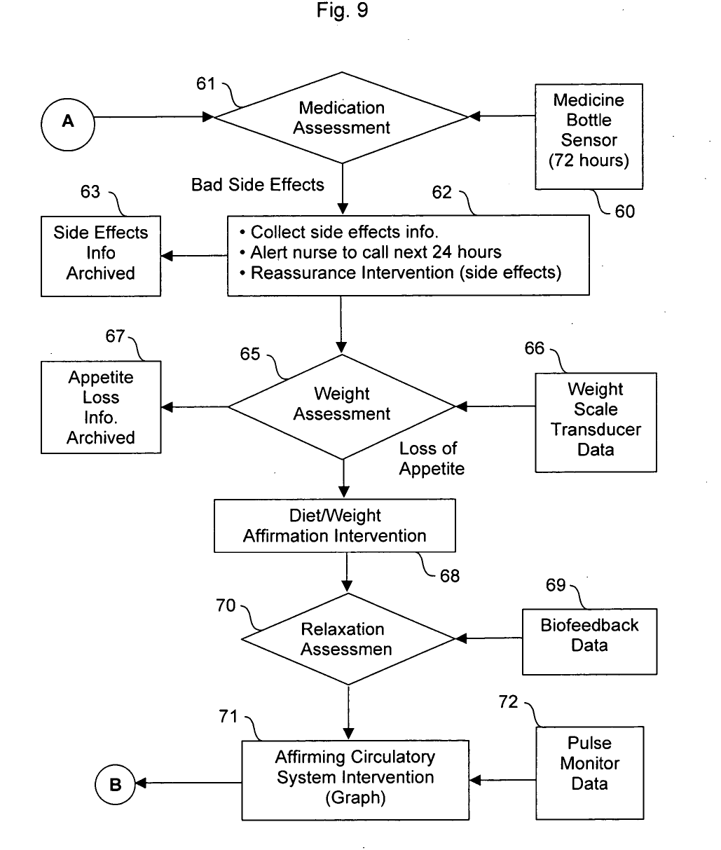

FIG. 9 illustrates the next set of interactions with the patient.

The information received from the Medicine Bottle sensor 15 that monitors the patient's compliance with the physician's drug regimen is downloaded 60 from the patient's in-home Data Hub prior to initiating the voice call. The program notes that the patient has not been taking her medication consistently over the last week. Therefore, an assessment 61 is made of the patient's non-compliance.

The patient reports that she has experienced “side effects.” The system then queries 62 to collect more details on the side effects of the medication. This information is archived 63. In addition, a nurse will contact the patient in the next 24 hours to see what adjustments in medications can be made.

The program then initiates a reassurance intervention 64 about the medication's side effects (“I'm sure we will find the right medication for you. You will not have to struggle with the side effects. Glad you brought it to our attention . . . ”). The goal of the intervention is to affirm the patient's desire to take the medications she needs without bad side effects, without shaming or criticizing her for non-compliance.

Next, the system asks about the patient's weight 65 (“You've actually lost weight—two pounds this week—do you know why?”). The query is based on data 66 collected by the weight scale transducer 10 and transmitted to the Installation.

The patient reports that she has lost some appetite (“I'm not as hungry”). This information is immediately archived 67. The system processes oral or coded collected data on the food labels of items she has consumed recently to query and reassure the patient that the weight loss may be due to “intelligent food choices.” This Diet/Weight Affirmative Intervention 68 seeks to reinforce positive food choices that the patient has been making.

Using the biofeedback data 69 downloaded from the patient's home Data Hub to the system computer, the program prompts a query about relaxation 70 exercises that the patient follows to reduce pain (“Is the ‘breath-watching’ helping you control the pain?”). The patient indicates that the exercises are helping her manage the pain.

The system then provides an Affirming Circulatory System Intervention 71, using data 72 provided by the pulse monitor 7. Using data from the last 30 days, the program generates a graph showing the steady improvement in the patient's pulse and blood pressure. The recorded message also lauds the patient for her successes.

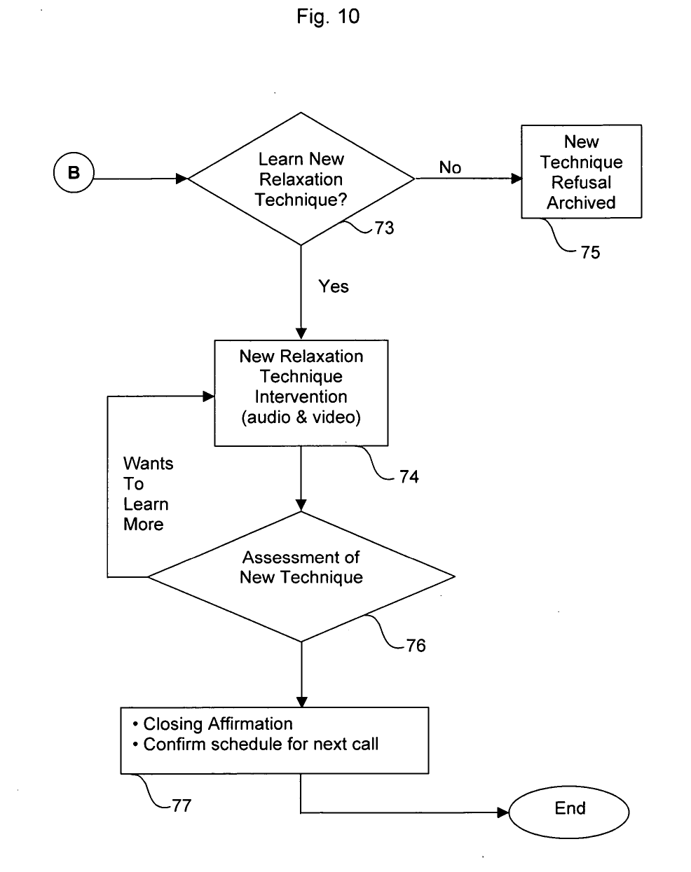

FIG. 10 illustrates the final phase of this telephone consultation with the patient. Based on a number of indicators of receptivity and relevance, the program prompts with a query, “Do you want to learn a brand new version of ‘Breath-watching’ right now?” 73.

When the patient replies in the affirmative, the system takes the patient through a series of steps to learn the new relaxation technique 74. A negative answer would have been archived 75 for future use. At each point, the program probes for feedback from the patient. The patient is rewarded with a relaxing image on the screen of her cell phone of a beach scene or other pleasant and relaxing clip, downloaded live from the Internet and transmitted to her phone.

An assessment 76 of the effect of the relaxation technique is performed.

The consultation terminates 77 with an affirmation of the patient's efforts to manage her pain and follow her physician's regimen. The schedule for the next phone consultation is confirmed.

The patient replies that she has experienced “side effects.” The system then queries 62 to collect more details on the side effects of the medication. This information is archived 63. In addition, a nurse will contact the patient in the next 24 hours to see what adjustments in medications can be made.

The program then initiates a reassurance intervention 64 about the medication's side effects (“I'm sure we will find the right medication for you. You will not have to struggle with the side effects. Glad you brought it to our attention . . . ”). The goal of the intervention is to affirm the patient's desire to take the medications she needs without bad side effects, without shaming or criticizing her for non-compliance.

Next, the system asks about the patient's weight 65 (“You've actually lost weight—two pounds this week—do you know why?”). The query is based on data 66 collected by the weight scale transducer 10 and transmitted to the Installation.

The patient reports that she has lost some appetite (“I'm not as hungry”). This information is immediately archived 67. The system processes oral or coded collected data on the food labels of items she has consumed recently to query and reassure the patient that the weight loss may be due to “intelligent food choices.” This Diet/Weight Affirmative Intervention 68 seeks to reinforce positive food choices that the patient has been making.

Using the biofeedback data 69 transmitted from the patient's home to the system computer, the program prompts a query about relaxation 70 exercises that the patient follows to reduce pain (“Is the ‘breath-watching’ helping you control the pain?”). The patient indicates that the exercises are helping her manage the pain.

The system then provides an Affirming Circulatory System Intervention 71, using data provided by the pulse/heart rate monitor 72. Using data from the last 30 days, the program generates a graph showing the steady improvement in the patient's pulse and blood pressure. The recorded message also lauds the patient for her successes.

FIG. 10 illustrates the final phase of this telephone consultation with the patient. Based on a number of indicators of receptivity and relevance, the program prompts with a query, “Do you want to learn a brand new version of ‘Breath-watching’ right now?” 73.

When the patient replies in the affirmative, the system takes the patient through a series of steps to learn the new relaxation technique 74. A negative answer would have been archived 75 for future use. At each point, the program probes for feedback from the patient. The patient is rewarded with a relaxing image on the screen of her cell phone of a beach scene or other pleasant and relaxing clip, downloaded live from the Internet and transmitted to her phone.

An assessment 76 of the effect of the relaxation technique is performed.

The consultation terminates 77 with an affirmation of the patient's efforts to manage her pain and follow her physician's regimen. The schedule for the next phone consultation is confirmed.

The patient replies that she has experienced “side effects.” The system then queries 62 to collect more details on the side effects of the medication. This information is archived 63. In addition, a nurse will contact the patient in the next 24 hours to see what adjustments in medications can be made.

The program then initiates a reassurance intervention 64 about the medication's side effects (“I'm sure we will find the right medication for you. You will not have to struggle with the side effects. Glad you brought it to our attention . . . ”). The goal of the intervention is to affirm the patient's desire to take the medications she needs without bad side effects, without shaming or criticizing her for non-compliance.

Next, the system asks about the patient's weight 65 (“You've actually lost weight—two pounds this week—do you know why?”). The query is based on data 66 collected by the weight scale transducer 10 and transmitted to the Installation.

The patient reports that she has lost some appetite (“I'm not as hungry”). This information is immediately archived 67. The system processes oral or coded collected data on the food labels of items she has consumed recently to query and reassure the patient that the weight loss may be due to “intelligent food choices.” This Diet/Weight Affirmative Intervention 68 seeks to reinforce positive food choices that the patient has been making.

Using the biofeedback data 69 transmitted from the patient's home to the system computer, the program prompts a query about relaxation 70 exercises that the patient follows to reduce pain (“Is the ‘breath-watching’ helping you control the pain?”). The patient indicates that the exercises are helping her manage the pain.

The system then provides an Affirming Circulatory System Intervention 71, using data provided by the pulse/heart rate monitor 72. Using data from the last 30 days, the program generates a graph showing the steady improvement in the patient's pulse and blood pressure. The recorded message also lauds the patient for her successes.

FIG. 10 illustrates the final phase of this telephone consultation with the patient. Based on a number of indicators of receptivity and relevance, the program prompts with a query, “Do you want to learn a brand new version of ‘Breath-watching’ right now?” 73.

When the patient replies in the affirmative, the system takes the patient through a series of steps to learn the new relaxation technique 74. A negative answer would have been archived 75 for future use. At each point, the program probes for feedback from the patient. The patient is rewarded with a relaxing image on the screen of her cell phone of a beach scene or other pleasant and relaxing clip, downloaded live from the Internet and transmitted to her phone.

An assessment 76 of the effect of the relaxation technique is performed.

The consultation terminates 77 with an affirmation of the patient's efforts to manage her pain and follow her physician's regimen. The schedule for the next phone consultation is confirmed.

Whereas the previous section provides a “broad brush” sketch of a typical telephone consultation, this section considers a single diagnostic/intervention sequence which links the severity and importance parameters in Table 2 to a specific case.

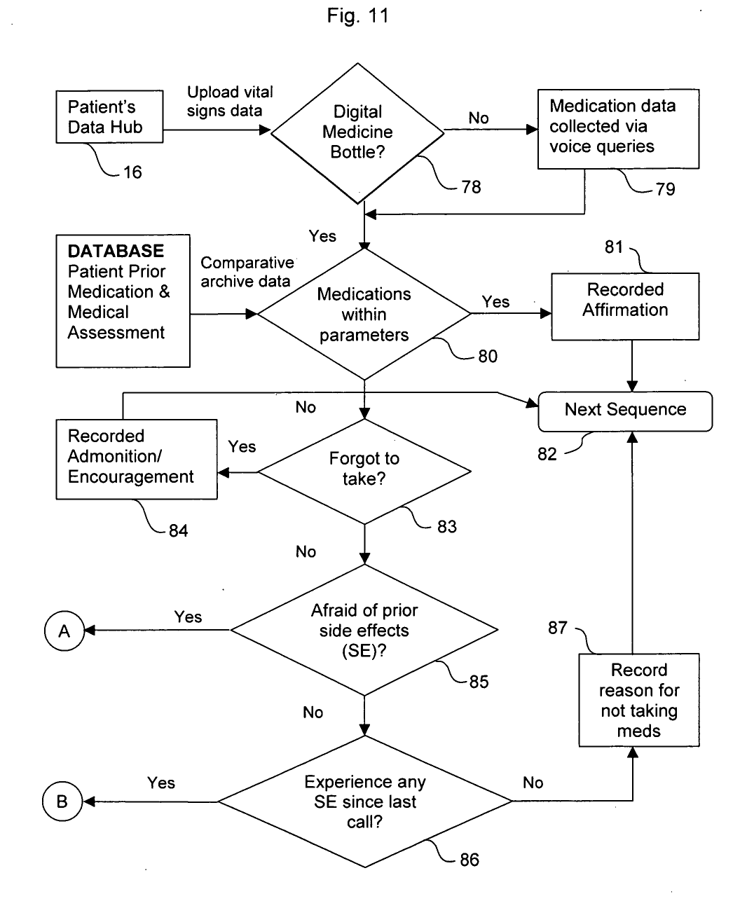

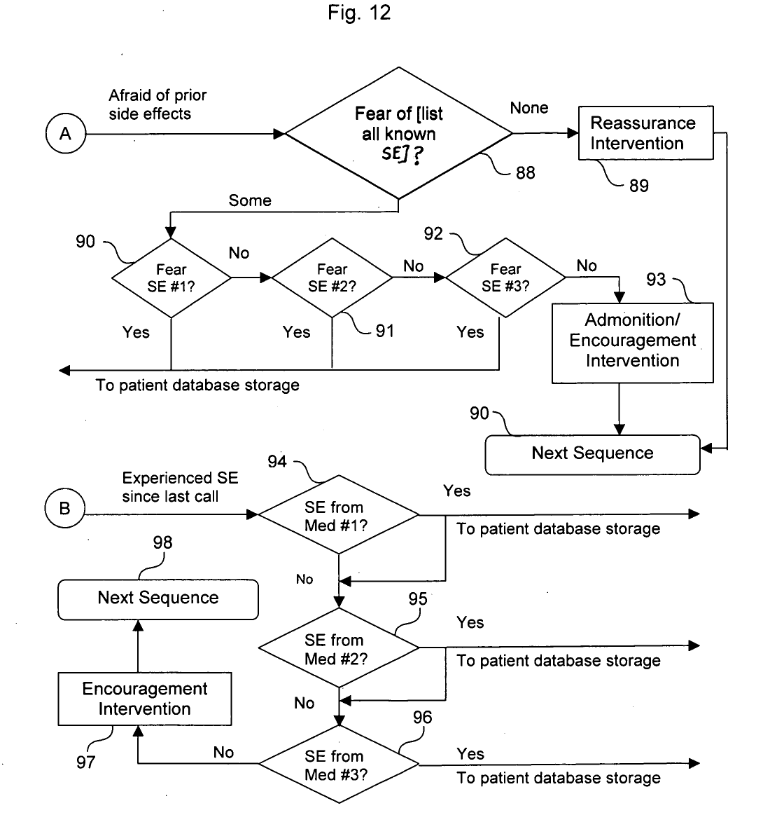

FIGS. 11 and 12 provide flowcharts for the detailed assessment of the patient's compliance with the medication regimen prescribed by her doctor. They illustrate the processes represented in the Medication Assessment 61 shown in FIG. 9. The first query by the system is to determine if the patient's sensor Data Hub 16 is collecting medication data digitally 78, by testing the operation of the Medicine Bottle sensor 15. If not, the program collects the information through a series of voice queries 79.

The system then checks the patient's prior medication records and medical assessment data to determine if the patient's medication usage is within the parameters 80 prescribed by her doctor. If yes, system provides a Recorded Affirmation 81 to reinforce the desired behavior and moves on to the next sequence 82 in the phone consultation. If no, the system assesses the reasons why the patient is not taking her medications. The first query asks if the patient simply forgot 83 to take her medications. If yes, the system provides an “admonition” 84 that takes the form of encouragement to comply with the doctor's regimen.

If the patient did not forget to take her medications, the system inquires about fears 85 associated with any prior side effects experienced from the medication. If the patient reports no fear of experiencing any prior side effects, the system inquires about any actual side effects 86 from the medications experienced since the last phone consultation. If the patient indicates no to this query as well, the system triggers the recording of a message 87 from the patient, which the counselor will review at a later time.

FIG. 12 displays the subroutines followed if the patient expresses fears of prior side effects or has experienced any actual side effects since the last consultation. If the patient was fearful of side effects, the program retrieves a list of all known side effects of the particular medication that the patient is taking, and presents it to the patient 88. If none of the known side effects are worrying the patient, the system reassures the patient that her concerns are unfounded, using a Reassurance Intervention 89. The program then prompts the Next Sequence 90 in the consultation, as dictated by the Script Generator 27.

If the patient expresses some fear of side effects 90-92, the system queries about all known side effects for the focal medication. For each side effect, the system queries whether that side effect is worrisome to the patient. Once all the side effects have been reviewed, the program responds with an Admonition/Encouragement Intervention 93. The patient's responses to the side effect probes are stored in the patient's database. The program then prompts the Next Sequence 90 in the script.

Sometimes, the patient stops taking her medications because of perceived or actual side effects. When the patient so indicates, the system inquires about the specific side effects for each medication that the patient's doctor has prescribed 94-96. Information about each side effect, whether positive or negative, is recorded in the patient's database. The program follows up with an Encouragement Intervention 97 and then prompts for the Next Sequence 98.

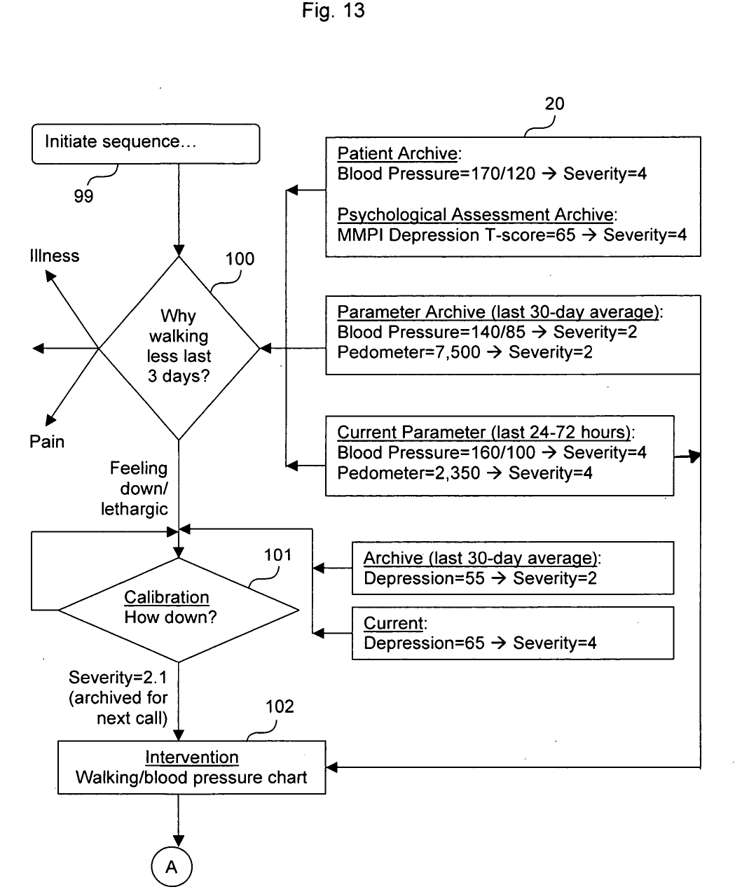

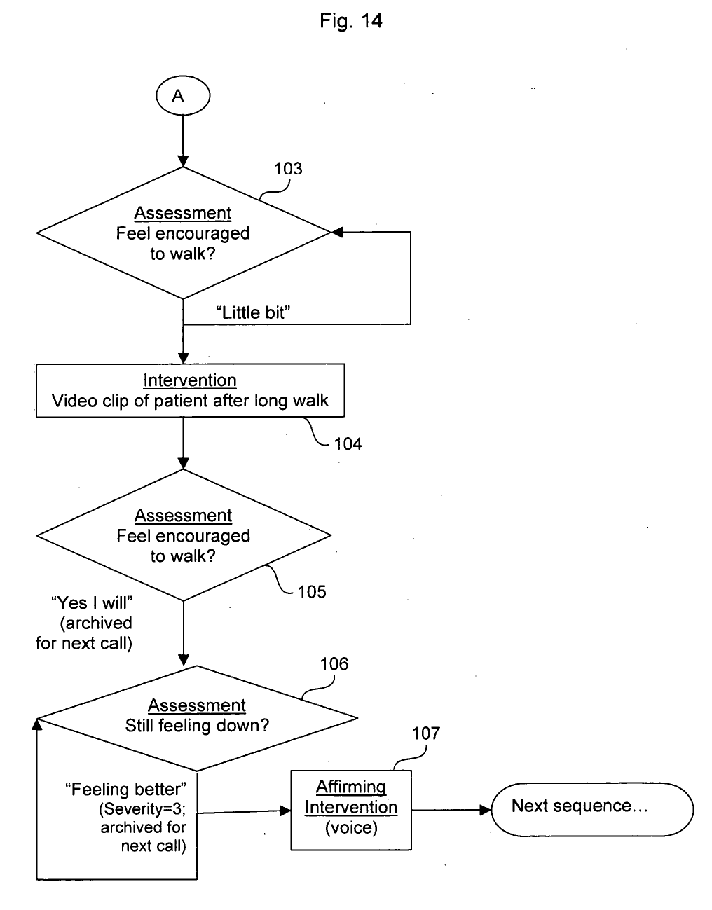

As shown in FIGS. 13 and 14, the system typically conducts an assessment of the effectiveness or efficacy of each intervention. In this example, the sequence is initiated by the program because of a shift in two interrelated diagnostics: blood pressure and walking exercise. From the Collected Information Library 20, the program recognizes that this patient suffered from high blood pressure (170/120) at the time of the medical assessment, which has a severity score of 4 according to Table 2. Blood pressure is an important indicator of health (importance coefficient=90; see Table 2) and this patient's blood pressure was dangerously high (severity=4 per Table 2) at the time of intake. However, the parameters collected from the blood pressure sensor 8 in the patient's home indicate a marked improvement in the patient's blood pressure. Over the 30 days prior to the telephone consultation, average blood pressure for the patient was 140/85, generating severity score of 2 indicating a minor risk.

At the same time, the pedometer has reported that the patient has walked an average of 7,500 steps a day over the last 30 days, which is in keeping with the exercise regimen prescribed by her physician.

During the last 24-72 hours, the sensors have indicated a significant drop in the patient's walking regimen and a concomitant increase in blood pressure. Although walking is not heavily weighted by itself with an importance coefficient, exercise is key to reducing the patient's blood pressure. Average blood pressure is 160/100 over the last 24-72 hours, generating a severity coefficient score of 4-a significant health risk. Daily walking has dropped from the 30-day average of 7,500 strides to 2,350.

The diagnostic/intervention sequence is initiated 99 by the program which uses a recorded query asking why she has reduced her walking 100 over the last three days. The patient replies that she has been “feeling a little depressed,” adding that she feels “too lethargic to walk.” The program then asks her to rate her depression 101 on a one to five scale. Note that this response is just one of many that the patient might provide. As indicated in FIG. 13, other reasons for not walking might include, but are not limited to, illness and pain. In response to each of these replies, the program would initiate a different set of follow-up queries and interventions 102.

The response from the patient (“a little depressed”) is not precise enough to permit the program to assign a severity score to the patient's level of depression. As in many diagnostic sequences, the program prompts with more detailed questions and assesses the new information from the patient against prior replies to zero in on a proper severity score for the patient's current level of depression.

Depression is an important health diagnostic with an importance coefficient of 60. Severe depression is life threatening in its own right e.g., suicidal ideation. In this case, depression is not that severe with a coefficient of 4; but the patient's low mood is interfering with the exercise regimen that her physician prescribed and the consequence is an increase in her blood pressure.

The program then initiates a therapeutic intervention: a graph that displays the relationship between the patient's walking and her blood pressure. This intervention is based on the principles of positive psychology, framed in a manner that shows the patient the positive consequences of following her walking regimen and its positive impact on her high blood pressure. This is communicated to the patient via a recorded message or, while she views the graph.

As shown in FIG. 14, the program queries whether seeing the chart has encouraged the patient to resume her walking regimen 103. She replies that she feels a “little bit” motivated to do more walking. As with other replies, the patient's natural-language response may require additional queries to assess the patient's level of motivation to resume walking. Alternatively, the system may ask the patient to use the one-to-five scale to assess her desire to increase her walking regimen. Based on the patient's responses, the program assesses the motivation as insufficient to bring about the desired change in behavior. Therefore, a second intervention is initiated.

The second intervention 104 consists of a video clip that the patient made of herself after returning from a long walk. The transmission of the video clip was prompted by the system program, which asked the patient to make a 60-second recording of herself immediately after a long walk to “journal how you feel physically and psychologically after a nice walk.” In the video clip, the patient tells how she feels physically right after a long walk (“My whole body feels more alive”), as well as her mood state (“It's like a cloud has lifted”). After the video clip, the program uses a prerecorded voice message from the patient's counselor to remind the patient how much better she feels after a walk, as well as its physical benefits.

At this point, the program conducts another assessment 105 of the patient's motivation to resume her walking regimen. The patient's reply (“Yes, I will.”) is archived for use in the next phone call or calls.

The shift in motivation 106 may also indicate an overall shift in the patient's mood state. The patient reports that she is “feeling better.” Several queries are required to calibrate the severity of the patient's depression. These queries generate a depression severity score of 1. This information is archived for use in subsequent phone calls.

The program closes this particular diagnostic/intervention sequence with a prerecorded message 107 from the patient's System counselor praising the patient for her commitment to increasing her walking, suggesting that her mood is also likely to improve if she resumes her previous walking regimen. As noted, this is but one sequence in a number of sequences in a typical System telephone consultation.

The functions represented by the various blocks in FIG. 3, as well as those illustrated by the flow chart of FIGS. 4, 6 and 8-14 are implemented by the computer programs running the Central Processing Unit 17 add the installation 4.

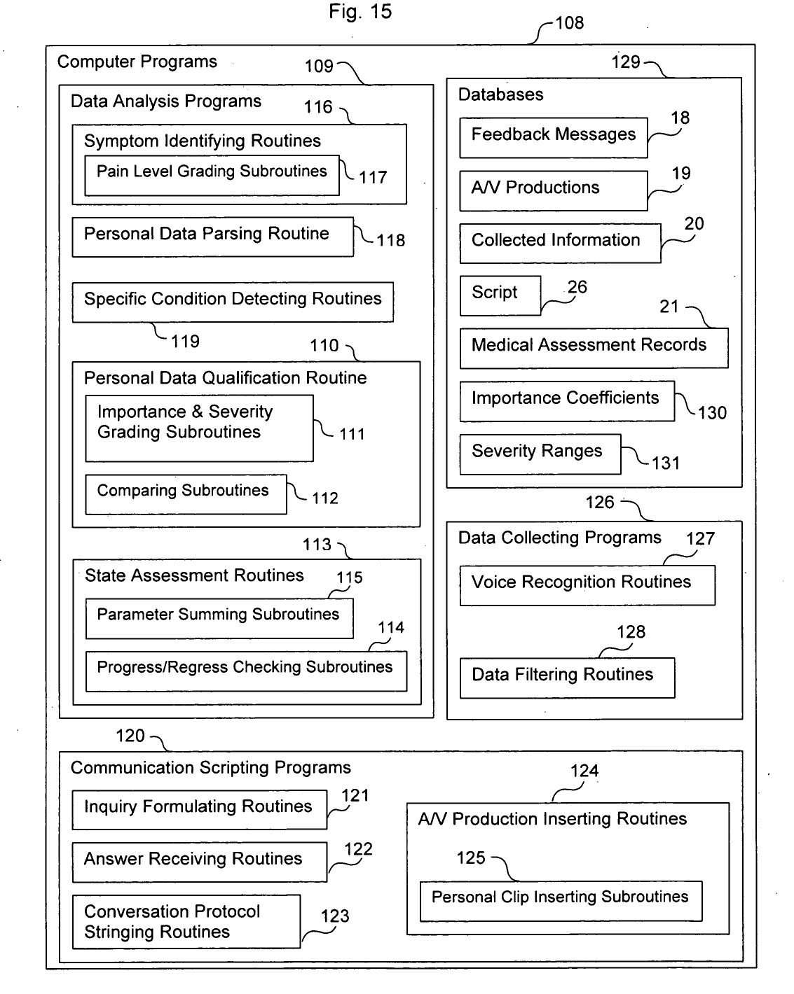

As illustrated in FIG. 15, the various programs 108 comprise embedded routines and subroutines.

The Data Analysis Programs 109 implement the Comparator 32, the Vital Sign Limit Generator 33, and the Data Analyzer 34. These programs comprise Personal Data Quantification routines 110 that include the Importance and Severity Grading subroutines 111. These subroutines assign the coefficients to the received personal parameters using the Importance Coefficient look-up table 130 and the Severity Range look-up table 131. The Data Analysis programs also include the received data Comparing subroutines 112, and the physical and mental State Assessment routines 113. The latter include the Progress/Regress Checking subroutines 114 and the Parameter Summing subroutines 115.

The Data Analysis Programs also include Symptom Identifying routines 116 which incorporate the Pain level Grading subroutines 117. Also included in the Data Analysis Programs are the Personal Data Parsing routines 118 and the Specific Condition Detecting routines 119. The former separates the psychological parameters from the physiological parameters.

The Communication Scripting programs 120 implement the Script Generator 27 and the Call Scheduler 28, and incorporate the Inquiry Formulating routines 121, the Answer Receiving routines 122, the Conversation Protocol Stringing routines 123, and the Audiovisual Production Inserting routines 124. The latter incorporate the Personal Clip Inserting subroutines 125.

The Data Collecting programs 126 include the Voice Recognition routines 127 and the Data Filtering routines 128. The latter implement the Communication Filter 30, and the Incoming Data/Info Switch 31.

The database or memory files 129 comprise the Feedback Message Library 18, the Audiovisual Production Library 19, the Collected Information Library 20, the Medical Assessment Records 21, the Script Library 26, the Importance Coefficient look-up table 130, and the Severity Range look-up table 131.

Several features of this diagnostic/intervention sequence are worth noting. First, the patient's medical and psychological conditions are treated as dynamic. In the described sequence, the system program drew on the patient's physiological and psychological assessment at intake, the patient's 30-day averages for key diagnostics, and finally the patient's 72-hour diagnostics. Thus, the current personal data obtained from the patient or from the monitoring devices are evaluated as a function of previously received, analyzed information stored in the Collected Information Library 20.

Second, the program is interactive, using the computerized telephone consultation to flesh out the bare-bones data automatically collected by sensors and transmitted to the system computer. The sensors indicate the existence of the problem, by taking the weight and severity of each diagnostic and comparing the pattern in the data against established templates (e.g., reduction in exercise leads to an increase in blood pressure). However, the sensors cannot diagnose why the patient has stopped exercising. Therefore, the program uses a series of queries over the telephone to develop a more comprehensive diagnosis.

Third, the program learns more about the patient through each diagnostic/intervention sequence. That is, the diagnostics and the interventions become more sophisticated over time, based on what has worked (and not worked) in past consultations. Fourth, the technology is highly transparent to the patient. The use of natural language probes and interventions from the system virtual counselor and the sophisticated voice-recognition system of the program permits the simulation of a truly interactive conversation between the patient and the artificial intelligence of the system program.

While the exemplary embodiments have been described, modifications can be made and other embodiments may be devised without departing from the spirit of the invention and the scope of the appended claims.

What is claimed is:

1. An automated system for vicariously and remotely monitoring the condition of a person and for coaching said person into beneficial behavior and activities, said system comprising:
a central automatic data processing installation;
at least one remote station accessible to said person;
at least one condition-monitoring apparatus associated with said station;
a communication link between said installation and said station;
wherein said central data processing installation includes a data collector for receiving personal data about said person from said station;
said data collector comprising a data receiver; and
a series of computer programs controlling said automatic data processing installation, including data analysis programs for determining the physical and psychological states of said person, and communication scripting programs.

2. The system of claim 1, wherein said automatic data processing installation further comprises a feedback messages library and a Collected Information library.

3. The system of claim 2, wherein said data analysis programs comprise program routines responsive to said data receiver for detecting specific symptoms in said person.

4. The system of claim 3, wherein said data analyzing programs further comprise program routines for comparing data received from said station to preset limits.

5. The system of claim 3, wherein said communication scripting programs comprise program routines for selectively retrieving at least one of said feedback messages as a function of said specific conditions.

6. The system of claim 5, wherein said library of feedback messages comprises a collection of audiovisual productions.

7. The system of claim 6, wherein:
said data communication link comprises a voice communication appliance;
said data collector comprises and oral information interpreter; and
said communication scripting programs further comprise program routines for formulating inquiries, program routines responsive to said oral information interpreter to accept answers from said person, and program routines for inserting at least one of said productions within said colloquy.

8. The system of claim 7, wherein said data analysis program further comprises program routines, responsive to said data collector, for evaluating said personal data as a function information in said collected information library.

9. The system of claim 8, wherein said monitoring apparatus comprises a series of sensors and transducers.

10. The system of claim 9, wherein said station further comprises an encoder for translating output signals from at least one of said series of sensors and transducers into digitally coded physiological diagnostic parameters.

11. The system of claim 10, wherein said person consists of a patient affected with at least one ailment; and said series of sensors and transducers comprises vital sign measurement devices.

12. The system of claim 11, wherein said data analysis program routines further comprise program routines, responsive to said oral information interpreter, to identify at least one psychological diagnostic parameter.

13. The system of claim 12, wherein said data analysis programs routines further comprise a look-up table for assigning importance and severity coefficients to said parameters.

14. The system of claim 13, wherein said data analysis programs further comprise program routines for summing a plurality of said parameters to generate a program branching command.

15. The system of claim 14, wherein said data analysis programs further comprise program routines for identifying at least one symptom.

16. The system of claim 15, wherein said symptom identifying routines includes subroutines for eliciting pain level grading from said person.

17. The system of claim 1, wherein said data analysis programs further comprise program routines for processing sais personal data to determine said physical state as a function of diagnostic parameters collected by said apparatus.

18. The system of claim 17, wherein:
said data communication link comprises a voice communication appliance; and
said data analysis programs further comprise program routines for processing said personal data to determine said mental state as a function of diagnostic parameters collected via said appliance.

19. The system of claim 15, wherein said data analysis programs further comprise formulae for calculating assessment codes as a function of said parameters and said pain level grading.

20. The system of claim 19, wherein said formulae comprise:
A=PS+PM+PL
$$PS = {\sum }_{php 1}^{phpx} Ic \times Sc , and PM = {\sum }_{psp 1}^{pspx} Ic \times Sc$$
where: A=Assessment Code
PS=Physical State
PM=Mental State
PL=Pain Level
php=physical parameter
psp=psychological parameter
Ic=Importance Coefficient
Sc=Severity Coefficient

21. The system of claim 20, wherein said data analysis programs further comprise program routines for updating previously recorded ones of said symptoms and assessment codes in said Collected Information library.

22. The system of claim 19, wherein said formulae include a sub-formula for calculating a physical state determinant as a function of said physiological diagnostic parameters, importance coefficients and severity coefficients.

23. The system of claim 22, wherein said formula includes a sub-formula for calculating a mental state determinant as a function of said psychological diagnostic parameters, importance coefficients and severity coefficients.

24. The system of claim 23, wherein said program routines for formulating inquiries comprise subroutines for eliciting from said person a pain level indication.

25. The system of claim 24, wherein said formula combines said physiological state determinant, said psychological state determinant and said pain level indication.

26. The system of claim 20, wherein said communication scripting programs comprise program routines for selecting a program branching as a function of one of said assessment codes.

27. The system of claim 26, wherein said data analysis programs further comprise program routines for determining progressive and regressive changes in said assessment code over a period of time.

28. The system of claim 27, wherein a plurality of said program routines for selectively retrieving comprise program subroutines responsive to said changes.

29. The system of claim 5, wherein said data communication link comprises a personal computer.

30. The system of claim 7, wherein said data communication link comprises a cell phone.

31. A method, for vicariously and remotely monitoring the condition and activities of a person, and for coaching said person into beneficial behavior and activities, which comprises the steps of:
providing a central automatic data processing installation and at least one remote station accessible to said person, said installation and station being linked over a communication network;
equipping said station with a plurality of vital sign and personal activity monitoring and information-providing devices and a data-gathering hub connected to said network;
recording and storing at said installation, a variety of advisory and therapeutic messages;
accumulating information provided by said devices at said installation;
analyzing said information to identify critical symptoms and conditions of said person; and
selectively retrieving and transmitting to said station, at least one of said messages in response to said analyzing.

32. The method of claim 31 which further comprises:
entertaining two-way communications with said person at said installation; and
interpreting said communication to detect therein pertinent personal information about said person.

33. The method of claim 32, wherein said step of interpreting comprises:
asking focused questions to said person and offering a limited number of simple specific answers to said questions; and
translating one of said answers received from said person into automatically processible data.

34. The method of claim 33, wherein said two-way communications comprise oral communications and said step of translating comprises voice recognizing said answer to generate said processible data.

35. The method of claim 34, wherein said messages comprise audio messages.

36. The method of claim 33, wherein said two-way communications comprise manually entered communications, and said step of translating comprises decoding coded textual information into digitally processible data.

37. The method of claim 36, wherein said messages comprise textual messages.

38. The method of claim 33, wherein said step of analyzing comprises:
parsing said information into physiological and psychological parameters;
checking at least one of said parameters against a preset safety limit; and
generating an alarm signal when said limit is exceeded by said parameters.

39. The method of claim 38, wherein said step of analyzing further comprises:
assigning to each of said parameters an importance coefficient;
dividing the span of each of said parameters into severity ranges and assigning a severity coefficient to each of said ranges;
for each parameter, combining said importance and severity coefficients to generate a symptom factor; and
totaling symptom factors of a plurality of parameters to generate a physical and mental assessment code for said person.

40. The method of claim 39 which further comprises using said parameters, factors, and code to generate program branching instructions.

41. A method, for vicariously and automatically providing a person with coaching advice in response to recorded indications of said person's condition, which comprises:
providing a computerized installation and a remote station equipped with condition-measuring and reporting devices, linked by a communication network;
recording and storing installation, a plurality of beneficial messages responsive to a plurality of personal status and conditions;
receiving at said installation, communications of personal data from said devices and person; and
analyzing said personal data to detect critical conditions and generate program branching instructions to selectively retrieve at least one of said messages.

42. The method of claim 41, wherein said step of analyzing comprises:
passing said personal data into physiological and psychological parameters; and
checking at least one of said parameters against a preset limit.

43. The method of claim 41, wherein said step of analyzing further comprises:
dividing the span of a plurality of said parameters into ranges, and assigning a severity coefficient in increasing order to said ranges;
assigning an importance coefficient to said plurality of said parameters; and
for each of said plurality of parameters, combining said coefficients to generate a condition assessment code for said person.

44. The method of claim 43, wherein said step of analyzing further comprises using said parameters and assessment codes to generate program branching instructions.

45. The method of claim 44 which further comprises:
scripting a plurality of conversational exchanges between said installation and said person;
submitting to said person focused inquiries;
suggesting a limited number of responses to each of said inquiries; and
stringing together a plurality of said conversational exchanges according to responses received from said installation and said branching instructions.
